(19) United States
(12) Patent Application Publication
(10) Pub. No.: US 2004/0047885 A1
(43) Pub. Date: Mar. 11, 2004

(54) Use of compounds which make it possible to modify the physicochemical properties of the skin and/or the mucous membranes as agents preventing or reducing the adhesion of microorganisms to the latter

(75) Inventors: Sylvie Cupferman, L'Hay Les Roses (FR); Geraldine Lerebour, Athis-Mons (FR); Veronique Guillou, Antony (FR); Pascal Simon, Vitry Sur Seine (FR)

Correspondence Address: NIXON & VANDERHYE, PC, 1100 N GLEBE ROAD, 8TH FLOOR, ARLINGTON, VA, 22201-4714, US

(73) Assignee: L'OREAL

(21) Appl. No.: 10/613,723

(22) Filed: Jul. 7, 2003

Related U.S. Application Data
(63) Continuation of application No. 09/782,521, filed on Feb. 14, 2001, now abandoned.

(30) Foreign Application Priority Data
Feb. 15, 2000 (FR) 00 01841

(51) Int. Cl. A61K 7/00

(52) U.S. Cl. 424/401

(57) ABSTRACT

The present invention relates to the use, as active ingredient, in cosmetic compositions or for the preparation of pharmaceutical compositions, of an effective quantity of at least one compound free of carbohydrate units, modifying the physicochemical properties of the surface of the skin and/or of the mucous membranes, so as to prevent or reduce the adhesion of microorganisms to the skin and/or the mucous membranes, chosen so that the decimal logarithm of the mean number of viable bacteria adhering to the epidermis, after a test consisting in bringing the said epidermis into contact with the test compound for 2 hours at 37° C., is at least 0.3 less than that obtained by a test carried out with water under the same conditions.

The invention relates to the use of compounds which make it possible to modify the physicochemical properties of the surface of the skin and/or the mucous membranes in a cosmetic composition or for the preparation of a pharmaceutical composition as agents preventing or reducing the adhesion of microorganisms, particularly bacteria, to the skin and/or the mucous membranes.

The human skin is permanently populated by a multitude of different microorganisms (bacteria, yeasts and fungi). The resident microbial flora, which is essential for good skin health, consists mainly of staphylococci (Staphylococcus epidermis and Staphylococcus hominis), corynebacteria, propionibacteria which are Gram+ such as Propionibacterium acnes, as well as a fungal flora mainly composed of Pytosporum ovale.

Skin infections are most often due to the disruption of the ecological balance among the resident flora following colonization of the skin by pathogenic exogenous microorganisms or following abnormal proliferation of an endogenous strain. The best known pathogenic microorganisms are Pseudomonas aeruginosa (Gram− ) which is responsible for small spots, folliculitis, red blotches and pruritus, Candida albicans which can cause inflammation of the corner of the lips, skin candidiasis, pruritus, folliculitis and aphtha, Staphylococcus aureus which can cause spots, folliculitis, impetigo and furuncles, and Streptococcus of group A responsible for impetigo.

To combat these microorganisms, it is common to use antibiotics or bactericides. The use of these compounds poses, nevertheless, the problem of nonspecificity of action affecting indiscriminately the pathogenic flora and the resident flora, and the problem of the risk of appearance of bacterial resistance, as well as problems of skin tolerance (irritations, allergies and the like).

It is also known to reduce or prevent the colonization of surfaces such as the teeth, the skin and/or the mucous membranes, by pathogenic microorganisms by preventing their attachment to these supports. The compounds used as antiadhesion agents described in the prior art are carbohydrates and derivatives of carbohydrates (WO 96 23 479, EP 380 084, U.S. Pat. No. 5,002,759, U.S. Pat. No. 4,859,656, WO 81 03 175, WO 93 14 773, WO 95 15 149, WO 95 07 084 and WO 95 17 898).

However, most carbohydrates constitute a source of carbon for bacteria and fungi. Their presence in cosmetic compositions consequently promotes microbial proliferation and requires increasing the concentration of preservatives (bactericides or bacteriostats). This disadvantage thus outweighs the benefit of the approach consisting in replacing antiobiotic or bactericidal compounds with compounds reducing microbial adherence.

The applicant has found, surprisingly, that a group of particular compounds, free of hydrocarbon units, made it possible to significantly reduce microbial adherence to the skin and/or the mucous membranes and to thus prevent the proliferation of potentially pathogenic microorganisms in the absence of antibiotic, bactericidal or fungicidal agents.

These compounds, unlike carbohydrates which bind to the microbial receptors to prevent bindings to the glycolipids of the corneocytes, act on the physicochemical properties of the surface of the skin and/or the mucous membranes, these physicochemical properties involving electrodynamic interactions due to Van der Waals forces, Lewis-type acid-base interactions and electrostatic interactions.

In addition, these compounds are not bactericidal. Because of this, they do not cause undesirable side effects on the skin and/or the mucous membranes.

The compounds according to the invention, when used as active ingredients, make it possible to reduce or prevent the adhesion of a microorganism whose overall surface charge is negative or positive by increasing respectively the negative or positive charge on the skin, so as to cause repulsion between the skin and/or the mucous membranes and the microorganism.

The compounds according to the invention, when used as active ingredients, make it possible, in addition, to reduce or prevent the adhesion of a microorganism by limiting as much as possible the Van der Waals type interactions between the skin and/or the mucous membranes and the microorganism, by promoting the repulsive interactions of the Lewis acid-base type and by limiting the attractive interactions of the Lewis acid-base type between the microorganism and the skin and/or the mucous membranes.

The expression to prevent or to reduce the adhesion of microorganisms should be understood to mean that the compound or the composition containing it may be used both preventively, for its capacity to completely or partially prevent the adhesion of microorganism, and curatively for its capacity to facilitate the detachment of the microorganisms.

These compounds are chosen so that the decimal logarithm of the mean number of viable bacteria adhering to reconstructed epidermis, after a test consisting in bringing the said epidermis into contact with the test compound for 2 hours at 37° C., is at least 0.3 less than that obtained by a test carried out with water under the same conditions.

The reconstructed epidermis used in the test indicated above is reconstructed human epidermis, equivalent to human skin, sold by EPISKIN.

This test makes it possible to evaluate the modifications in the physicochemical properties of the surface of the skin and/or of the mucous membranes, involving Van der Waals electrodynamic interactions, Lewis-type acid-base interactions and electrostatic interactions.

The subject of the invention is therefore the use, as active ingredient, in a cosmetic composition or for the preparation of a pharmaceutical composition, of an effective quantity of at least one compound free of carbohydrate units, modifying the physicochemical properties of the surface of the skin and/or of the mucous membranes, so as to prevent or reduce the adhesion of microorganisms to the skin and/or the mucous membranes, chosen so that the decimal logarithm of the mean number of viable bacteria adhering to reconstructed epidermis, after a test consisting in bringing the said epidermis into contact with the test compound for 2 hours at 37° C., is at least 0.3 less than that obtained by a test carried out with water under the same conditions.

Preferably, compounds will be used for which the above-defined decimal logarithm is 0.5 less than and more particularly 1 less than that of water under the same conditions.

The experimental protocol which makes it possible to choose these compounds will be defined below.

The nature of these compounds may be completely different and they may be chosen, by way of nonlimiting example, from nonsolid fatty substances at room temperature, polymers, surfactants and/or mixtures thereof.

The compounds used according to the invention may very well be hydrophilic or lipophilic.

There is preferably used, as active ingredient in a cosmetic composition or for the preparation of a pharmaceutical composition, an effective quantity of compounds free of carbohydrate units modifying the physicochemical properties of the surface of the skin and/or of the mucous membranes chosen from surfactants such as disodium cocoamphodiacetate, oxyethylenated glyceryl cocoate (7 EO) such as the product sold by COGNIS under the name Cetiol HE, PEG-20 hexadecenyl succinate, PEG-15 stearyl ether; the ricinoleic monoethanolamide monosulphosuccinate salts such as the product sold by Goldschmidt under the name REWODERM S1333, oxyethylenated hydrogenated ricinoleic triglyceride containing 60 ethylene oxide units such as the product sold by Nikko under the name NIKKOL HCO-60 or such as the product sold by BASF under the name CREMOPHOR RH60, polymers such as Poloxamers, which are block copolymers of ethylene oxide and propylene oxide, such as for example the product sold under the name Lutrol F68 by BASF and Poloxamer 407 sold under the name SYNPERONIC PE/F 127 by UNIQEMA; polyacrylamides such as polyacrylamide/C13-14 Isoparaffin/Laureth-7 such as the product sold by SEPPIC under the name SEPIGEL 305. The nonsolid fatty substances at room temperature (that is to say at a temperature ranging from about 20 to 35° C. such as sesame oil, sweet almond oil, apricot stone oil, sunflower oil, octoxyglyceryl palmitate (or 2-ethylhexyl glyceryl ether palmitate) such as the product marketed under the name Mexanyl GP by the company Chimex, octoxyglyceryl behenate (or 2-ethylhexyl glyceryl ether behenate), dioctyl adipate, tartrate of branched C₁₂-C₁₃ dialcohols such as the product sold under the name Cosmacol ETI by Enichem.

According to the invention, the compound(s) or the composition containing them are used for topical application to the skin and/or the mucous membranes.

The adhesion of microorganisms to the skin and/or the mucous membranes has consequences which range from mere unpleasantness (odour, small spots and the like) to more serious or less serious diseases.

One of the aspects of the invention is therefore to propose the use of a compound free of carbohydrates as active ingredient in a cosmetic composition or for the preparation of a pharmaceutical composition.

In particular, the subject of the invention is the cosmetic use by topical application of at least one compound as active ingredient in a cosmetic composition intended to reduce bad body odours and/or intended for body hygiene health care.

The expression body hygiene health care is understood to mean any substance or preparation intended to be brought into contact with various superficial parts of the human body and/or with the teeth and/or the mucous membranes so as to clean them, protect them, maintain them in good condition, modify the appearance thereof, perfume them and correct the odour thereof.

In particular, the subject of the invention is the cosmetic use by topical application of at least one compound as active ingredient in a cosmetic composition intended to combat comedones and/or dandruff.

The microbial flora of the surface of the skin is responsible for a large number of disorders.

Thus, the subject of the invention is also the use of at least one compound as active ingredient for the preparation of a pharmaceutical composition intended to be used by topical application to combat mycosis and/or acne, particularly juvenile acne.

The quantity of compound which can be used according to the invention quite obviously depends on the desired effect and should be a quantity effective for partially or completely preventing adhesion of microorganisms or for facilitating the detachment of microorganisms.

By way of example, the quantity of compound used according to the invention may range, for example, from 0.1 to 100%, preferably from 0.5 to 50% and more particularly from 1 to 25% of the total weight of the composition.

The subject of the invention is also a cosmetic method for treating disorders linked to the adhesion of microorganisms consisting in applying to the skin a cosmetic composition comprising at least one compound according to the invention in a cosmetically acceptable medium.

The expression cosmetically acceptable medium is understood to mean a medium compatible with the skin, the scalp, the mucous membranes, the nails and the hair.

The cosmetic and pharmaceutical compositions used according to the invention may be provided in all the galenic forms normally used for topical application, in particular in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, it being possible for these spherules to be polymeric nanoparticles such as nanospheres and nanocapsules, or even better, lipid vesicles of ionic and/or nonionic type.

When the composition according to the invention comprises a fatty phase, the latter preferably represents from 1 to 60% of the total weight of the composition.

This fatty phase may comprise one or more oils preferably chosen from the group consisting of:

volatile or nonvolatile silicones which are linear, branched or cyclic, organomodified or otherwise, water-soluble or fat-soluble,

mineral oils such as paraffin oil and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grapeseed oil, rapeseed oil, copra oil,

synthetic oils such as isoparaffins,

fluorinated and perfluorinated oils,

fatty acid esters.

They may also comprise, as fatty substances, one or more fatty alcohols, fatty acids or waxes (paraffin, polyethylene wax, Carnauba wax, beeswax).

In a known manner, the compositions used in the invention may, in addition, contain customary adjuvants in the cosmetic field such as gelling agents and/or hydrophilic or lipophilic conventional thickening agents; hydrophilic or lipophilic active agents; preservatives, solvents; antioxidants; perfumes; emulsifiers; moisturizing agents; pigmenting agents; depigmenting agents; keratolytic agents; vitamins; emollients; sequestrants; surfactants; polymers; alkalinizing or acidifying agents; fillers; anti-free radical agents; ceramides; sun screens (in particular ultraviolet-screening agents); insect repellents; slimming agents; colouring matter; anti-dandruff agents.

The quantities of these various adjuvants are those conventionally used in the fields considered.

Of course, persons skilled in the art will be careful to choose the possible compound(s) to be added to the composition according to the invention such that the advantageous properties intrinsically attached to the composition in accordance with the invention are not, or not substantially, adversely modified by the addition envisaged.

As solvents, there may be mentioned hydrophilic organic solvents, and for example linear or branched lower monoalcohols having from 1 to 8 carbon atoms such as ethanol, propanol, butanol, isopropanol, isobutanol; polyethylene glycols having from 6 to 80 ethylene oxides, polyols such as propylene glycol, isoprene glycol, butylene glycol, glycerol; mono- or dialkyls of isosorbide in which their alkyl groups have from 1 to 5 carbon atoms such as dimethyl isosorbide; glycol ethers such as diethylene glycol monomethyl or monoethyl ether and ethers of propylene glycol such as dipropylene glycol methyl ether.

The organic solvents may represent from 5 to 98% of the total weight or the composition.

The compositions used in the present invention may be fluid to a greater or lesser degree and may have the appearance of a white or coloured cream, an ointment, a milk, a lotion, a serum, a paste, a foam or a solid.

They may be optionally applied to the skin in aerosol form.

They may be provided in solid form, and for example in the form of a stick.

They may be used as health care product, as cleansing product for the skin or the hair, as sun screen product, as make-up product such as foundations, lipsticks, mascaras, blushers, and/or as simple deodorant product.

Thus, the subject of the invention is a cosmetic health care, cleansing, make-up or deodorant composition comprising at least one compound according to the invention.

The antiadhesion test corresponds to the protocol below:

Before bacterial adhesion, the reconstructed epidermis is brought into contact for 2 hours with 25 mg of the product to be tested at 37° C. 1 ml of bacterial suspension of Staphylococcus aureus at a concentration of 107 microorganisms/ml in Tryptone salt is then added thereto. After incubating for 24 hours at 37° C., the bacterial suspension is emptied and five rinsings are carried out with 1 ml of sterile distilled water. The epidermis, detached from its support, is then ground with the aid of a food processor in 18 ml of Tryptone salt. A decimal dilution is carried out on this suspension in Tryptone salt, and 1 ml of the dilution is then inoculated into 15 ml of Trypticase Soy agar and the medium is incubated for 24 hours at 37° C. The adherent and viable cells are then counted.

This antiadhesion test makes it possible to evaluate the efficacy of molecules alone or of finished products.

Before the antiadhesion test, the following viability test is carried out:

A bacteria/test product mixture, in the same ratio as in the antiadhesion test is brought into contact for 24 hours at 37° C. The test may require incubation, with stirring, in order to avoid the death of the bacteria through lack of oxygen, in particular as regards fats which are not solid at room temperature. The microorganisms are counted by decimal dilution in Tryptone salt and inoculated with a 100 _l scraper on Trypticase Soy agar. The colonies are counted after 24 hours of incubation at 37° C.

The test for viability carried out prior to the antiadhesion test makes it possible to rule out any bactericidal component for the molecules or the finished products tested and to demonstrate only the antiadhesion activity.

The following examples present the results obtained for various compounds tested according to the invention and a particular embodiment of a composition according to the invention.

These examples are of course given by way of illustration and have absolutely no limitative character.

### EXAMPLES OF COMPOUNDS USED ACCORDING TO THE INVENTION

The results obtained for the compounds presented here result from the use of the protocol detailed above.

The figures presented opposite the compound correspond to the reduction of the decimal logarithm of the mean number of viable Staphylococcus aureus adhering to reconstructed epidermis after treatment with the compound under the conditions defined by the preceding test compared with the decimal logarithm of the mean number of viable Staphylococcus aureus adhering to reconstructed epidermis after treatment with water under the same conditions.

| | |
|---|---|
| Disodium cocoamphodiacetate | 1.34 |
| Oxyethylenated glyceryl cocoate (7 EO) | 1.41 |
| Ricinoleic monoethanolamide monosulpho- | 3.84 |
| succinate salt (tested at 5% active | |
| substance) | |
| Oxyethylenated (600 E) hydrogenated ricinoleic | 0.8 |
| triglyceride sold under the name NIKKOL | |
| HCO-60 by NIKKO (tested at 10% active | |
| substance) | |
| Poloxamer 407: copolymer of ethylene | 0.81 |
| oxide, propylene oxide and ethylene | |
| oxide (98 EO/67 PO/98 EO) (MW: 12 000) | |
| sold under the name: SYNPERONIC PE/F 127 | |
| by UNIQEMA | |
| Polyacrylamide/C13-14 Isoparaffin/ | 1.43 |
| Laureth-7 (tested at 1% active | |
| substance) sold under the name SEPIGEL | |
| 305 by SEPPIC | |
| Tartrate of branched C₁₂-C_{13 dialcohols} | 2.31 |
| Apricot stone oil | 0.81 |
| Dioctyl adipate | 0.90 |

### EXAMPLE OF COMPOSITION USED TO ACCORDING TO THE INVENTION

W/O Emulsion for Face Care:

| | |
|---|---|
| Oxyethylenated polymethylcetyl dimethyl | 3% |
| methylsiloxane (ABIL EM 90 ® from | |
| GOLDSCHMIDT) | |
| Palmitate of 2-ethylhexyl glyceryl ether | 10% |
| (MEXANYL GP ® from CHIMEX) | |
| Tartrate of branched Chd 12-C₁₃ dialcohols | 10% |
| (COSMACOL ETI ® from ENICHEM) | |
| Oxyethylenated glyceryl cocoate (7 EO) | 3% |
| (CETIOL HE ® from GOGNIS) | |
| Condensate of ethylene oxide, propylene | 3% |
| oxide and ethylene oxide (MW: 8 350) | |
| (75 EO/30 PO/75 EO) (LUTROL F 68 ® from | |
| BASF) | |
| Water | QS 100 |
| Antioxidant | qs |
| Perfume | qs |

After treatment with the above composition, under the conditions defined by the preceding test, a decrease of 3.96 in the decimal logarithm of the mean number of viable Staphylococcus aureus adhering to reconstructed epidermis is observed compared with the decimal logarithm of the mean number of viable Staphylococcus aureus adhering to the reconstructed epidermis after treatment with water under the same conditions.

1. Use, as active ingredient, in a cosmetic composition or for the preparation of a pharmaceutical composition, of an effective quantity of at least one compound free of carbohydrate units, modifying the physicochemical properties of the surface of the skin and/or of the mucous membranes, so as to prevent or reduce the adhesion of microorganisms to the skin and/or the mucous membranes, chosen so that the decimal logarithm of the mean number of viable bacteria adhering to reconstructed epidermis, after a test consisting in bringing the said epidermis into contact with the test compound for 2 hours at 37° C., is at least 0.3 less than that obtained by a test carried out with water under the same conditions.

2. Use, as active ingredient, in a cosmetic composition or for the preparation of a pharmaceutical composition, of an effective quantity of at least one compound according to claim 1, characterized in that the decimal logarithm of the mean number of viable bacteria adhering to reconstructed epidermis, after a test consisting in bringing the said epidermis into contact with the test compound for 2 hours at 37° C., is at least 0.5 less than that obtained by a test carried out with water under the same conditions.

3. Use, as active ingredient, in a cosmetic composition or for the preparation of a pharmaceutical composition, of an effective quantity of at least one compound according to either of claims 1 and 2, characterized in that the decimal logarithm of the mean number of viable bacteria adhering to reconstructed epidermis, after a test consisting in bringing the said epidermis into contact with the test compound for 2 hours at 37° C., is at least 1 less than that obtained by a test carried out with water under the same conditions.

4. Use according to any one of claims 1 to 3, characterized in that the active ingredient is chosen from surfactants, polymers and nonsolid fatty substances at room temperature.

5. Use according to any one of claims 1 to 4, characterized in that the active ingredient is chosen from disodium cocoamphodiacetate, oxyethylenated glyceryl cocoate (7 EO), the ricinoleic monoethanolamide monosulphosuccinate salts, oxyethylenated hydrogenated ricinoleic triglyceride containing 60 ethylene units, Poloxamers, polyacrylamides, PEG-20 hexadecenyl succinate, sesame oil, octoxyglyceryl palmitate, octoxyglyceryl behenate, dioctyl adipate, PEG-15 stearyl ether, apricot stone oil, tartrate of branched C₁₂-C₁₃ dialcohols and mixtures thereof.

6. Use according to any one of claims 1 to 5, characterized in that the active ingredient is present in a quantity ranging from 0.1% to 100% of the total weight of the composition.

7. Use according to claim 6, characterized in that the active ingredient is present in a quantity ranging from 0.5% to 50% of the total weight of the composition.

8. Use according to either of claims 6 and 7, characterized in that the active ingredient is present in a quantity ranging from 1% to 25% of the total weight of the composition.

9. Use according to any one of claims 1 to 8, characterized in that the composition is provided in the form of a lotion, an aqueous gel, a serum, an emulsion or a dispersion of lipid vesicles.

10. Method of cosmetic treatment for treating disorders linked to the adhesion of microorganisms consisting in applying to the skin and/or the mucous membranes a cosmetic composition comprising at least one compound as defined according to any one of claims 1 to 5 in a cosmetically acceptable medium.

11. Cosmetic use by topical application of at least one compound as defined according to any one of claims 1 to 5 in a cosmetic composition intended to reduce bad body odours and/or intended for body hygiene health care.

12. Cosmetic use by topical application of at least one compound as defined according to any one of claims 1 to 5 in a cosmetic composition intended to combat comedones and/or dandruff.

13. Use of at least one compound as defined according to any one of claims 1 to 5 for the preparation of a pharmaceutical composition intended to be used by topical application to combat mycosis and/or acne.
